(19) United States
(12) Patent Application Publication
(10) Pub. No.: US 2021/0177376 A1
(43) Pub. Date: Jun. 17, 2021

(54) GUIDEWIRE ULTRASOUND (US) PROBE FOR A MINIMALLY PERTURBING MEASUREMENT OF BLOOD FLOW IN BRAIN VESSEL

(71) Applicant: BIOSENSE WEBSTER (ISREAL) LTD., Yokneam (IL)

(72) Inventor: Assaf Govari, Haifa (IL)

(21) Appl. No.: 16/716,260

(22) Filed: Dec. 16, 2019

(51) Int. Cl. A61B 8/12; A61B 8/06; A61M 25/01; A61B 8/08; A61B 8/00

(52) U.S. Cl. CPC A61B 8/12; A61B 8/06; A61M 25/0127; A61M 2025/09108; A61B 8/461; A61B 8/5223; A61B 8/488

(57) ABSTRACT

A medical probe includes a guidewire, a magnetic position sensor, and an ultrasound (US) transducer. The guidewire is configured for insertion into a blood vessel of a patient. The magnetic position sensor is fitted at a distal end of the guidewire and is configured to produce signals indicative of a position of the distal end. The US transducer is fitted at the distal end of the guidewire and is configured to transmit US waves inside the blood vessel, and acquire respective US echoes indicative of blood velocity in the blood vessel.

### FIELD OF THE INVENTION

The present invention relates generally to medical devices, and particularly to probes for cerebrovascular applications.

### BACKGROUND OF THE INVENTION

Minimally invasive probes for vascular applications, including for cerebrovascular applications, were previously proposed in the patent literature. For example, U.S. Pat. No. 5,207,226 describes a device and method for measuring fluid flow within a vessel having a fluctuating elastic wall, such as, for example, an artery. The device includes a catheter, an internal stabilizer or frame for establishing an invariant cross-section in the vessel wall, e.g., by engaging the entire internal circumference of vessel wall in order to maintain a constant cross-sectional area of the vessel, and a fluid velocity detecting system, such as, for example, a Doppler crystal transducer. The method for measuring includes the steps of stabilizing the vessel wall and measuring the velocity of fluid passing through the stabilized cross-section of the vessel wall.

As another example, U.S. Pat. No. 6,704,590 describes a guiding catheter that includes a Doppler sensor disposed at a distal end of a flexible shaft. The Doppler sensor can sense a blood flow turbulence level within a chamber of the heart or a blood vessel of the heart. Detecting changes in a blood flow turbulence level is used to assist guiding of the distal end of the flexible shaft. The Doppler sensor may include a piezoelectric sensor or an optical sensor. The sensor readings may be processed to show turbulence through a time domain or frequency domain presentation of velocity. The sensor readings can be used to modulate an audible waveform to indicate turbulence. The guiding catheter may further include steering apparatus enabling deflection of the distal tip.

U.S. Patent Application Publication 2012/316419 describes a catheter based system for providing functional and morphological characterization of arteries, comprising a catheter configured for insertion in an artery, and a sensor system for mapping hemodynamic parameters mounted on the catheter, wherein the sensor system includes a plurality of probes comprising at least two anemometric probes spatially arranged in a deployed position and configured to measure flow velocity components in at least two different positions spaced apart in a direction orthogonal to the axial direction of the catheter.

### SUMMARY OF THE INVENTION

An embodiment of the present invention provides a medical probe including a guidewire, a magnetic position sensor, and an ultrasound (US) transducer. The guidewire is configured for insertion into a blood vessel of a patient. The magnetic position sensor is fitted at a distal end of the guidewire and is configured to produce signals indicative of a position of the distal end. The US transducer is fitted at the distal end of the guidewire and is configured to transmit US waves inside the blood vessel, and acquire respective US echoes indicative of blood velocity in the blood vessel.

In some embodiments, the guidewire, the magnetic position sensor and the US transducer jointly have a maximal diameter that does not exceed 3.0 mm.

In some embodiments, the US transducer is configured to transmit the US waves in a distal direction and receive the US echoes from the distal direction. In other embodiments, the US transducer is configured to transmit the US waves in a proximal direction and receive the US echoes from the proximal direction.

In an embodiment, the magnetic position sensor is formed on a flexible printed-circuit-board wrapped around the distal end of the guidewire.

There is additionally provided, in accordance with an embodiment of the present invention, a medical system including a guidewire ultrasound (US) probe and a processor. The probe includes a guidewire, a magnetic position sensor, and an US transducer. The guidewire is configured for insertion into a blood vessel of a patient. The magnetic position sensor is fitted at a distal end of the guidewire and is configured to produce signals indicative of a position of the distal end. The US transducer is fitted at the distal end of the guidewire and is configured to transmit US waves inside the blood vessel, and acquire respective US echoes indicative of blood velocity in the blood vessel. The processor is configured to (a) receive from the US transducer electrical signals indicative of a Doppler shift of the echoes due to blood velocity, (b) analyze the electrical signals to derive the blood velocity, and (c) display the derived blood velocity to a user.

In some embodiments, the processor is configured to spectrally analyze the electrical signals to determine a maximal Doppler shift.

There is further provided, in accordance with an embodiment of the present invention, a manufacturing method including fitting a magnetic position sensor at a distal end of a guidewire for insertion into a blood vessel of a patient. An ultrasound (US) transducer is fitted to a distal end of the guidewire. The magnetic position sensor and the US transducer are wired.

There is furthermore provided, in accordance with an embodiment of the present invention, a method including inserting a guidewire into a blood vessel of a patient, the guidewire having a magnetic position sensor and an ultrasound (US) transducer fitted at a distal end thereof, wherein the magnetic position sensor is configured to acquire position signals, and wherein the US transducer is configured to transmit US waves inside the blood vessel, and acquire respective US echoes indicative of blood velocity in the blood vessel. The guidewire is navigated to a target location in the blood vessel using signals acquired by the magnetic position sensor. At the target location, electrical signals are received from the US transducer which are indicative of a Doppler shift of the echoes due to blood velocity. The electrical signals are analyzed to derive the blood velocity. The derived blood velocity is displayed to a user.

In some embodiments, the method further includes retracting the guidewire out of the blood vessel of the patient.

The present invention will be more fully understood from the following detailed description of the embodiments thereof, taken together with the drawings in which:

### BRIEF DESCRIPTION OF THE DRAWINGS

FIG. 1 is schematic, pictorial illustration of a cerebrovascular blood velocity measurement system, in accordance with embodiments of the present invention;

FIG. 2 is a side-view of the distal end of the hollow guidewire of FIG. 1 inside a blood vessel, in accordance with an embodiment of the present invention;

FIG. 3 is graph that schematically illustrates echo signal amplitude as a function of the Doppler frequency shift of the echo, in accordance with an embodiment of the present invention;

FIG. 4 is a flow-chart that schematically illustrates a manufacturing method of the guidewire US probe of FIG. 3, in accordance with an embodiment of the present invention; and

FIG. 5 is a flow-chart that schematically illustrates a method for measuring blood velocity in a blood vessel using the system of FIG. 1, in accordance with an embodiment of the present invention.

### DETAILED DESCRIPTION OF EMBODIMENTS

### Overview

An invasive probe comprising an ultrasound (US) transducer at its distal end may be inserted into a blood vessel to measure velocity of blood that flows in the vessel using the Doppler effect. The US reflecting media in blood are the red blood cells, and ultrasound waves reflected from the red blood cells (i.e., US echoes) change in frequency according to blood velocity relative to the US beam direction.

When the dominant direction of blood flow is exactly towards (or exactly away from) an US beam emitted by the transducer, the echoes gain a maximal positive (or negative) frequency shift compared to the frequency of the emitted US waves. Blood velocity, V_{B}, can be derived from the maximal Doppler frequency shift, Δf, via the equation V_{B}=V_{US}Δf/f, where V_{US} is the velocity of the US wave in blood and f is the US frequency. Since the Doppler effect is small, Δf may be estimated using spectral (e.g., Fourier) analysis of an echo signal acquired using sequences of pulsed US waves.

In practice, however, careful preparation of the intravascular measurement may be required, for example including, fixating the blood vessel walls to have well-defined blood flow profile. Such preparations can complicate the device and usage of an intravascular US blood velocity measurement method.

Embodiments of the present invention that are described hereinafter provide an ultrathin guidewire with a miniature US transducer fitted at its distal edge. The miniature US transducer is configured to emit US waves mainly at a distal and/or a proximal direction. The ultrathin guidewire and miniature transducer enable, and maintain, a largely laminar flow of the blood in the vicinity of the distal end. The transducer is configured to either emit ultrasound continuously or in pulses (A-mode), i.e., it is not an imaging transducer. The US transducer may comprise, for example, a miniature piezoelectric transceiver. A processor determines blood velocity based on analysis of the received echoes.

In some embodiments, the disclosed guidewire comprises a magnetic position sensor at its distal end, which is used to track a location of the distal end in the blood vessel where blood velocity is determined. The magnetic position sensor may comprise a single-, double-, or triple-axis magnetic transducer. The position of the distal end may be tracked, for example, using a magnetic position tracking system, such as the CARTO® system (made by Biosense-Webster, Irvine, Calif.).

In some embodiments, in order to fit the very small diameter guidewire the magnetic position sensor is formed on a flexible printed circuit board (PCB) wrapped around the ultrathin guidewire. An example of a magnetic position sensor formed on a flexible PCB, which is wrapped around a distal end of a sheath of a catheter, is described in U.S. patent application Ser. No. 16/248,393, filed Jan. 15, 2019, entitled “Position Sensor on Brain Clot Sheath and Location Pad Collar,” which is assigned to the assignee of the present patent application and whose disclosure is incorporated herein by reference.

In some embodiments, the maximal diameter of the US guidewire, including the US transducer and magnetic position sensor, does not exceed outer diameter (OD) of 3.0 mm.

The disclosed ultrathin US guidewire may be used to characterize blood flow before advancing a full-diameter catheter into a location in question, and may thereby simplify brain catheterization procedures.

### System Description

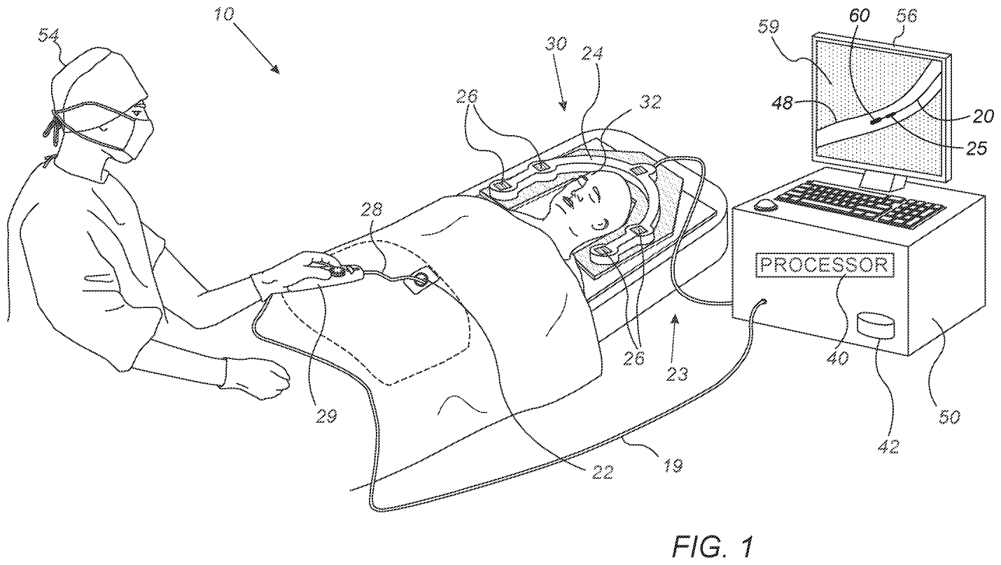

FIG. 1 is schematic, pictorial illustration of a cerebrovascular blood velocity and position measurement system 10, in accordance with embodiments of the present invention.

In some embodiments, prior to performing the disclosed diagnostic procedure, CT images of a patient 32 are acquired. The CT images are stored in a memory 42 for subsequent retrieval by a processor 40. The processor uses the CT images to present, for example, brain section image 59 demonstrating a blood vessel 48 in question on a monitor 56.

System 10 comprises a hollow guidewire 20, wherein a distal end of hollow guidewire 20 is inserted into patient 32 through a sheath 28, through an entry point 22 at an artery of a thigh of patient 32. Physician 54 navigates the distal end of hollow guidewire 20 through arteries to a brain location in question where blood velocity is to be measured.

To manipulate guidewire 20, physician 54 uses a controller handle 29, which is connected to the proximal end of hollow guidewire 20. The proximal end of controller handle 29 is coupled to a cable 19 that, in turn, is connected to a control console 50, to receive signals from sensors fitted at the distal end of guidewire 20.

During navigation, the position of a US transducer 60, which is fitted at a distal edge of hollow guidewire 20, is tracked using a magnetic tracking sub-system 23, which tracks position coordinates of magnetic sensor 25 fitted at the distal end of hollow guidewire 20.

Sub-system 23 comprises a location pad 24 that is fixed to the bed, and is fitted with magnetic field radiators 26 which are fixed in position relative to the head of patient 32 and which transmit alternating sinusoidal magnetic fields into a region 30 where the head of patient 32 is located. In response, magnetic sensor 25 generates position signals that are received by console 50.

During the procedure, the head of patient 32 is harnessed to keep it motionless. A location tracking system using a location pad similar to location pad 24 is described in U.S. patent application Ser. No. 15/674,380, filed Aug. 10, 2017,entitled “ENT Image Registration,” which is assigned to the assignee of the present patent application and whose disclosure is incorporated herein by reference with a copy provided in the Appendix.

Control console 50 comprises a processor 40, typically a general-purpose computer, with suitable front end and interface circuits (not shown) for receiving the US and position signals, as well as for controlling other components of system 10. Typically, processor 40 is configured to receive multiple measurements from US transducer 60 and to use these measurements to calculate blood velocity at the location of transducer 60.

Processor 40 uses software stored in a memory 42 to operate system 10. The software may be downloaded to processor 40 in electronic form, over a network, for example, or it may, alternatively or additionally, be provided and/or stored on non-transitory tangible media, such as magnetic, optical, or electronic memory. In particular, processor 40 runs a dedicated algorithm that enables processor 40 to perform the steps disclosed in FIG. 5.

### Guidewire US Probe for a Minimally Perturbing Measurement of Blood Flow in Brain Vessel

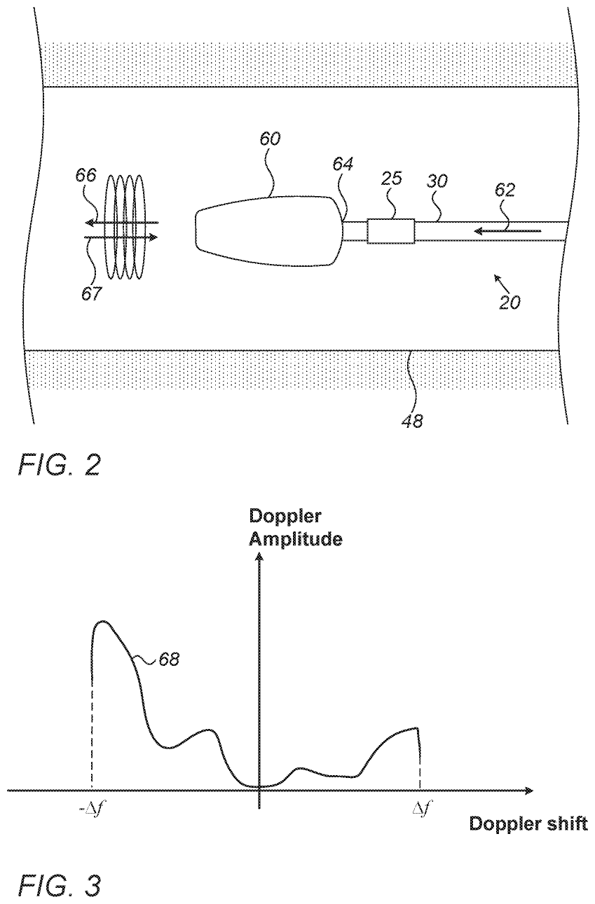

FIG. 2 is a side-view of distal end 30 of guidewire 20 of FIG. 1 inside blood vessel 48, in accordance with an embodiment of the present invention. Distal end 30 is seen after being extracted from sheath (which is not shown in this figure, for clarity). Hollow guidewire 20 has an exceptionally low diameter, on the order of several hundred microns, while still being stiff enough, as described below, to be inserted into a blood vessel of the brain.

US transducer 60, which is attached (64) to the distal edge of hollow guidewire 20, is navigated to a target location inside vessel 48 using magnetic position tracking sub-system 23 that tracks the location of magnetic position sensor 25. In the shown embodiment, US transducer 60 has an elongated shape so as not to disrupt blood flow. US transducer 60 is configured to emit US waves in a largely distal direction 66 parallel to a longitudinal axis 62 of distal end 30, and to receive Doppler shifted echoes 67 reflected from blood. However, other configurations are possible, in which, for example, transducer 60 is further configured to also emit US waves in a largely proximal direction and receive echoes from that direction. Typically, the lateral dimension of transducer 60 is up to 3.0 mm in diameter. The small dimension is enabled in part, due to transducer 60 being an A-mode transducer, in an embodiment.

In an embodiment, transducer 60 is fixed (e.g., glued) to the distal end of guidewire 20 over a distal perimeter of the hollow guidewire (not shown). In some embodiments, the wall of hollow guidewire 20 is made of a thin-wall polyimide tube reinforced with a metal coil. In an embodiment, the polyimide wall, or another suitable plastic, includes a braided coil of metal wire that serves to stiffen guidewire 20 while not limiting maneuverability.

As further seen, magnetic position sensor 25 is tightly wrapped around distal end 30, and glued together with its electrical leads (not shown). As noted above, in some embodiments, in order to conform with the small diameter guidewire, the magnetic position sensor is formed on a flexible printed circuit board (PCB) wrapped around ultra-thin hollow guidewire 20.

The example illustration shown in FIG. 2 is chosen purely for the sake of conceptual clarity. FIG. 2 shows only parts relevant to embodiments of the present invention. Additional elements, such as electrical wires, are omitted for clarity of presentation.

FIG. 3 is graph that schematically illustrates a spectrum 68 of the echo signal, i.e., the echo signal amplitude as a function of the Doppler frequency shift of the echo, in accordance with an embodiment of the present invention. Spectrum 68 is derived by processor 40 using Fourier analysis of signals from transducer 60 of FIG. 2.

As seen, spectrum 68, despite being smoothed by the processing algorithm, has complex features and is continuous between a negative cutoff Doppler frequency denoted −Δf and a positive cutoff Doppler frequency denoted +Δf. The reason for this shape is that, in practice, US echoes of variable strengths are received from multiple directions and not only from the distal and/or proximal ones. Yet, regardless of the shape of spectrum 68, processor 40 can accurately determine blood velocity, V_{B}, by estimating the cutoff values of the Doppler shift, −Δf and +Δf, and calculating V_{B} using the aforementioned equation for blood velocity, V_{B}=V_{US}Δf/f.

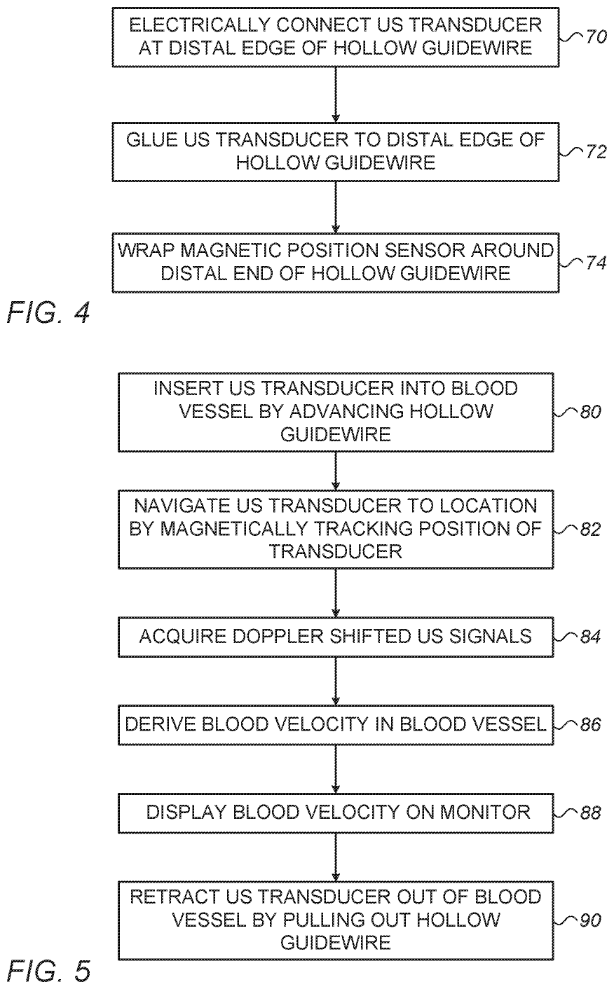

FIG. 4 is a flow-chart that schematically illustrates a manufacturing method for the guidewire US probe of FIG. 3, in accordance with an embodiment of the present invention. The process begins with electrically connecting miniature US transducer 60 to at a distal edge of hollow guidewire 20 to wires that run inside hollow guidewire 20, at a transducer wiring manufacturing step 70.

Next, at a transducer gluing manufacturing step 72, transducer 60 is glued (64) to hollow guidewire 20 over a perimeter of the distal edge hollow reinforced guidewire 20. Alternatively, heat may be used to melt proximal perimeters of a plastic shell of transducer 60 to the exterior guidewire surface. Finally, at a magnetic position sensor disposing step 74, magnetic position sensor 25 is wrapped around distal end 30 of hollow guidewire 20, and glued together with its electrical leads (not shown).

The example flow-chart shown in FIG. 4 is chosen purely for the sake of conceptual clarity. Additional steps, such as connecting electrical wires to sensor 25, are omitted to simplify presentation. A more detailed description of steps of manufacturing is omitted for simplicity.

FIG. 5 is a flow-chart that schematically illustrates a method for measuring blood velocity in a blood vessel using the system of FIG. 1, in accordance with an embodiment of the present invention. The medical procedure begins with physician 54 inserting transducer 60 into vessel 48 by advancing hollow guidewire 20, at a guidewire US probe insertion step 80.

Next, physician 54 uses system 10, including sensor 25, to navigate and position transducer 60 at a vessel 48 location in question, at a US transducer positioning step 82. Physician 54 then acquires Doppler shifted echoes, at a blood velocity measurement step 84.

The acquired US signals are processed by processor 40, which derives blood velocity at the location, at a blood velocity derivation step 86. Processor 40 displays the derived blood velocity to physician 54 on a monitor 56, at blood velocity displaying step 88.

Finally, physician 54 retracts transducer 60 from vessel 48 by pulling out hollow guidewire 20, at guidewire US probe retraction step 90.

Although the embodiments described herein mainly address cerebrovascular applications, the methods and systems described herein can also be used in other applications, such as in measuring blood velocity at any other sufficiently large blood vessel of the body.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical probe, comprising:
a guidewire for insertion into a blood vessel of a patient;
a magnetic position sensor, which is fitted at a distal end of the guidewire and is configured to produce signals indicative of a position of the distal end; and
an ultrasound (US) transducer, which is fitted at the distal end of the guidewire and is configured to transmit US waves inside the blood vessel, and acquire respective US echoes indicative of blood velocity in the blood vessel.

2. The medical probe according to claim 1, wherein the guidewire, the magnetic position sensor and the US transducer jointly have a maximal diameter that does not exceed 3.0 mm.

3. The medical probe according to claim 1, wherein the US transducer is configured to transmit the US waves in a distal direction and receive the US echoes from the distal direction.

4. The medical probe according to claim 1, wherein the US transducer is configured to transmit the US waves in a proximal direction and receive the US echoes from the proximal direction.

5. The medical probe according to claim 1, wherein the magnetic position sensor is formed on a flexible printed-circuit-board wrapped around the distal end of the guidewire.

6. A medical system, comprising:
a guidewire ultrasound (US) probe comprising:
a guidewire for insertion into a blood vessel of a patient;
a magnetic position sensor, which is fitted at a distal end of the guidewire and is configured to produce signals indicative of a position of the distal end; and
an ultrasound (US) transducer, which is fitted at the distal end of the guidewire and is configured to transmit US waves inside the blood vessel, and acquire respective US echoes indicative of blood velocity in the blood vessel; and
a processor, which is configured to:
receive from the US transducer electrical signals indicative of a Doppler shift of the echoes due to blood velocity;
analyze the electrical signals to derive the blood velocity; and
display the derived blood velocity to a user.

7. The medical system according to claim 6, wherein the guidewire, the magnetic position sensor and the US transducer jointly have a maximal diameter that does not exceed 3.0 mm.

8. The medical system according to claim 6, wherein the US transducer is configured to transmit the US waves in a distal direction and receive the US echoes from the distal direction.

9. The medical system according to claim 6, wherein the US transducer is configured to transmit the US waves in a proximal direction and receive the US echoes from the proximal direction.

10. The medical system according to claim 6, wherein the processor is configured to spectrally analyze the electrical signals to determine a maximal Doppler shift.

11. A manufacturing method, comprising:
fitting a magnetic position sensor at a distal end of a guidewire for insertion into a blood vessel of a patient;
fitting an ultrasound (US) transducer to a distal end of the guidewire; and
wiring the magnetic position sensor and the US transducer.

12. A method, comprising:
inserting a guidewire into a blood vessel of a patient, the guidewire having a magnetic position sensor and an ultrasound (US) transducer fitted at a distal end thereof, wherein the magnetic position sensor is configured to acquire position signals, and wherein the US transducer is configured to transmit US waves inside the blood vessel, and acquire respective US echoes indicative of blood velocity in the blood vessel; and
navigating the guidewire to a target location in the blood vessel using signals acquired by the magnetic position sensor;
at the target location, receiving from the US transducer electrical signals indicative of a Doppler shift of the echoes due to blood velocity;
analyzing the electrical signals to derive the blood velocity; and
displaying the derived blood velocity to a user.

13. The method according to claim 12, and comprising retracting the guidewire out of the blood vessel of the patient.
